(19) United States
(12) Patent Application Publication
(10) Pub. No.: US 2025/0107817 A1
(43) Pub. Date: Apr. 3, 2025

(54) MEDICAL DEVICE

(71) Applicant: TERUMO KABUSHIKI KAISHA, Tokyo (JP)

(72) Inventor: Kosuke NISHIO, Irvine, CA (US)

(73) Assignee: TERUMO KABUSHIKI KAISHA, Tokyo (JP)

(21) Appl. No.: 18/899,278

(22) Filed: Sep. 27, 2024

(30) Foreign Application Priority Data
Sep. 29, 2023 (JP) 2023-169508

(51) Int. Cl. A61B 17/22; A61B 17/00; A61B 17/32

(52) U.S. Cl. CPC A61B 17/22; A61B 17/00234; A61B 17/32; A61B 2017/00238; A61B 2017/00305; A61B 2017/00402; A61B 2017/00477; A61B 2017/00685; A61B 2017/22079; A61B 2217/005

(57) ABSTRACT

Provided is a medical device that removes an object in a body lumen, the medical device including: a rotatable drive shaft; a cutter that is rotationally driven by the drive shaft and is able to cut the object; an outer tube shaft that rotatably accommodates the drive shaft; and a loosely wound conveyance coil disposed to wrap around the outer periphery of the drive shaft. The conveyance coil is fixed to the drive shaft only at a distal end section of the conveyance coil.

### CROSS-REFERENCES TO RELATED APPLICATION

This application claims priority to Japanese Patent Application No. 2023-169508 filed on Sep. 29, 2023, the entire content of which is incorporated herein by reference.

### TECHNOLOGICAL FIELD

The present invention generally relates to a medical device for removing an object in a body lumen.

### BACKGROUND DISCUSSION

Treatment methods for treating a stenosed part due to plaque, thrombus, or the like in a blood vessel include, for example, a method for expanding the blood vessel with a balloon and a method for placing a meshed or coiled stent in the blood vessel as a support of the blood vessel. However, with these methods, it is difficult to treat a stenosed part hardened due to calcification or a stenosed part located in a bifurcation of a blood vessel. As a device that can provide treatment even in such a case, an atherectomy device that cuts a lesion and removes a cut object by suction has been proposed. An example of such a device is disclosed in U.S. Pat. No. 8,628,549.

The atherectomy device has a high-speed rotating drive shaft inside an outer tube. When a helical conveyance coil is attached to the drive shaft, the conveyance coil rotating with high-speed rotation of the drive shaft can convey the cut object to the outside of the body. That is, the conveyance coil that rotates along with the drive shaft can convey the cut object in the proximal direction based on the principle of an Archimedes pump.

### SUMMARY

The drive shaft of the atherectomy device receives a wide range of additional torque when, for example, a cutter cuts the lesion, the cutter does not cut the lesion, or the cutter bites into the lesion. The entire length of the drive shaft extends and contracts like a spring according to the torque load. The conveyance coil that needs to follow the extension and contraction of the drive shaft directly receives friction with the drive shaft and the outer tube shaft that rotatably accommodates the conveyance coil, and thus, cannot follow the drive shaft without a mechanical load. For this reason, when the conveyance coil and the drive shaft are weakly joined, the conveyance coil may be detached or separated from the drive shaft.

In particular, when a torque load is applied to the conveyance coil in a curved blood vessel in a living body, the conveyance coil is less likely to uniformly extend and contract entirely, and non-uniform extension and contraction occurs inside the outer tube. For this reason, a fixed portion of the conveyance coil with respect to the drive shaft continuously receives a mechanical load at all times, and may be broken in the end.

The medical device disclosed here can prevent a conveyance coil fixed to a drive shaft from being detached from the drive shaft.

The medical device is able to accomplish this by the aspect described in (1) below.

(1) The medical device disclosed here as one example of the medical device that removes an object in a body lumen includes: a drive shaft that is rotatable; a cutter that is rotationally driven by the drive shaft and is able to cut the object; an outer tube shaft that rotatably accommodates the drive shaft; and a conveyance coil that is loosely wound and is disposed to wrap around an outer periphery of the drive shaft, wherein the conveyance coil is fixed to the drive shaft only at a distal end section of the conveyance coil.

In the medical device according to (1), when the drive shaft extends and contracts in the axial direction, a section of the conveyance coil proximal to the distal end section fixed to the drive shaft can move in the axial direction relative to the drive shaft, so that a load on the fixed section of the conveyance coil with respect to the drive shaft can be reduced. Therefore, this medical device can prevent detachment of the conveyance coil fixed to the drive shaft from the drive shaft.

(2) In the medical device according to (1), during rotation, the conveyance coil may contract, and the drive shaft may extend. Due to the contraction of the conveyance coil, it is possible to prevent a fluid that receives a force from the conveyance coil from generating a swirl flow, whereby the conveyance efficiency can be improved. Due to the extension of the drive shaft, it is possible to suppress an occurrence of defects due to a decrease in length of the drive shaft.

(3) In the medical device according to (1) or (2), the conveyance coil may be fixed to the drive shaft only at a distal end of the conveyance coil. With this configuration, the load on the fixed section of the conveyance coil with respect to the drive shaft can be further reduced, whereby the detachment of the conveyance coil from the drive shaft can be effectively suppressed. In addition, the flexibility of the drive shaft and the conveyance coil is improved, and the operability of the medical device in the curved body is improved.

(4) In the medical device according to any one of (1) to (3), the conveyance coil may include a multi-wire coil formed by winding a plurality of wire materials, may include an integrated portion formed by melting and integrating a plurality of wire materials arranged in a circumferential direction over the entire circumference, and may be fixed to the integrated portion. When the section of the drive shaft to which the conveyance coil is fixed is not the integrated portion but any of the wire materials, the wire material may be broken. On the other hand, when the conveyance coil is fixed to the integrated portion of the drive shaft, damage to the drive shaft can be suppressed.

(5) In the medical device according to any one of (1) to (4), a coil proximal end located at a proximal end of the conveyance coil may be formed to have a curved surface. This configuration can prevent damage to the drive shaft and the outer tube shaft with which the coil proximal end of the conveyance coil is in contact.

According to another aspect, a medical device operable to remove an object in a body lumen comprises: a rotatable drive shaft that extends in an axial direction and that has a distal end portion; a cutter connected to the drive shaft so that rotation of the drive shaft results in rotation of the cutter, with the cutter being located at the distal end portion of the drive shaft and being configured to cut the object when the cutter is brought into contact with the object while rotating; an outer tubular shaft having an interior in which is located the drive shaft, the outer tubular shaft having an inner periphery facing the outer periphery of the drive shaft so that a space exists between the inner periphery of the outer tubular shaft and the outer periphery of the drive shaft; and an opening through which is conveyable a part of the object cut by the cutter and liquid in the body lumen. The opening is in communication with the space between the inner periphery of the outer tubular shaft and the outer periphery of the drive shaft so that the part of the object cut by the cutter and the liquid can enter the space by way of the opening. A conveyance coil is helically wound around the outer periphery of the drive shaft so that axially adjacent windings of the conveyance coil are axially spaced apart from one another, wherein the conveyance coil is located in the space, and wherein the conveyance coil has a distal end portion terminating at a distal-most end of the conveyance coil and a proximal end portion terminating at a proximal-most end of the conveyance coil. At least a part of the distal end portion of the conveyance coil is fixed to the drive shaft so that rotation of the drive shaft results in rotation of the conveyance coil so that the conveyance coil conveys the part of the object cut by the cutter and the liquid in the axial direction, and an entirety of the proximal end portion of the conveyance coil is a non-fixed portion of the conveyance coil that is not fixed to the drive shaft so that the drive shaft is movable relative to the non-fixed portion of the conveyance coil.

Another aspect of the disclosure involves a method comprising positioning a cutter adjacent an object in a lumen in a living body. The cutter is connected to a drive shaft, and the drive shaft is positioned in an outer tubular shaft, with a conveyance coil wound around an outer periphery of the drive shaft so that adjacent windings of the conveyance coil are spaced apart from one another. The conveyance coil has a distal end section and a proximal end section, and the conveyance coil is fixed to the drive shaft only at the distal end section of the conveyance coil so that an entirety of the proximal end section of the conveyance coil is a non-fixed portion of the conveyance coil that is not fixed to the drive shaft. The method also comprises rotating the cutter through rotation of the drive shaft while the cutter is in contact with the object to cut the object, with the rotation of the drive shaft resulting in rotation of the conveyance coil. The non-fixed portion of the conveyance coil that is proximal to the distal end section is movable in an axial direction relative to the drive shaft during the rotating of the cutter through the rotation of the drive shaft.

### BRIEF DESCRIPTION OF THE DRAWINGS

FIG. 1 is a plan view illustrating a medical device according to an embodiment.

FIG. 2 is a cross-sectional view illustrating a distal end part of the medical device.

FIG. 3 is a plan view illustrating an inner layer and an outer layer of the medical device in the vicinity of the distal end part, FIG. 3 including a plan view of a drive shaft and a conveyance coil and a cross-sectional view of other components.

FIGS. 4A-4C are diagrams including a cross-sectional view of an outer tubular shaft and a plan view of the drive shaft and the conveyance coil, in which FIG. 4A illustrates a state in which the drive shaft is rotated, FIG. 4B illustrates a state in which a helical pitch of the conveyance coil is reduced, and FIG. 4C illustrates a state in which the drive shaft is twisted due to a torque load being applied to the drive shaft.

FIG. 5 is a schematic diagram illustrating a state in which the medical device removes a lesion.

### DETAILED DESCRIPTION

An embodiment of the medical device disclosed here, representing one example of the new medical device, will be described below with reference to the drawings. The size and ratio of each member in the drawings may be exaggerated for convenience of description and may be different from the actual size and ratio. In addition, in the present specification, a side of a medical device 10 to be inserted into a living body is referred to as a “distal side” and a side to be operated is referred to as a “proximal side”.

The medical device 10 according to the present embodiment is inserted into a blood vessel in acute lower limb ischemia or deep-vein thrombosis, and used for a treatment including destroying (breaking-up or breaking-apart) and removing an object such as a thrombus, plaque, atheroma, and a calcified lesion. The object to be removed is not necessarily limited to a thrombus, plaque, atheroma, and a calcified lesion, and may be any object that can be present in the body lumen.

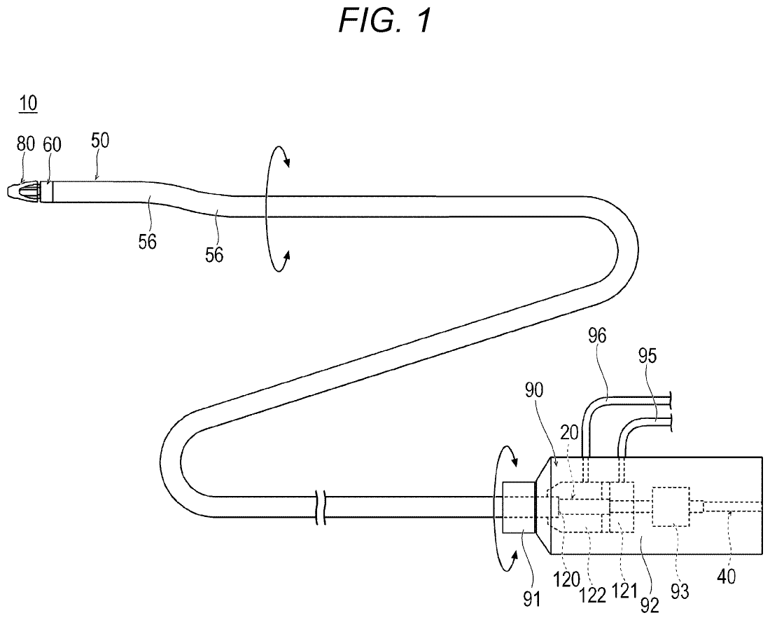

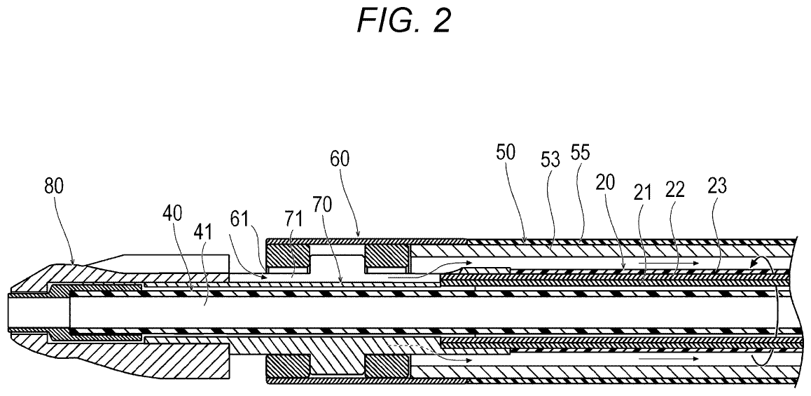

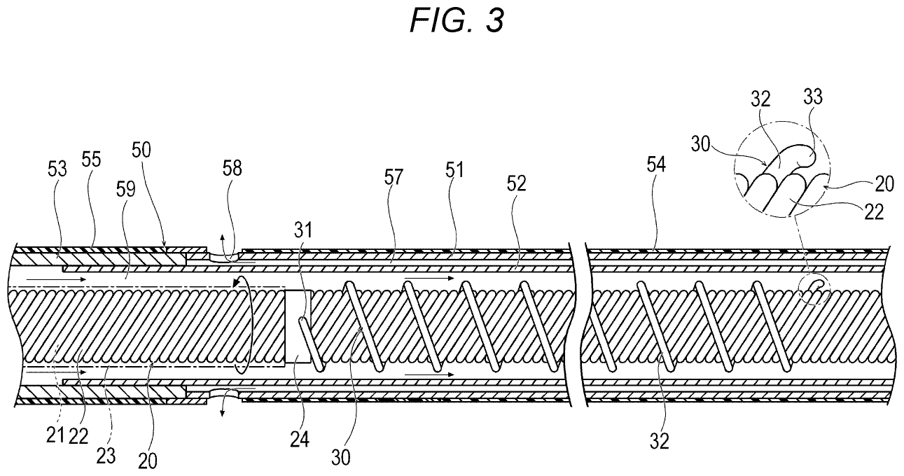

As illustrated in FIGS. 1 and 2, the medical device 10 includes an elongated drive shaft 20, a conveyance coil 30 (shown in FIG. 3 for example) that conveys an object, an outer tubular shaft 50 that accommodates the drive shaft 20 and the conveyance coil 30, a cutter 80 that cuts an object such as a thrombus, a rotation shaft 70 that connects the cutter 80 and the drive shaft 20, a distal bearing 60 that rotatably supports the rotation shaft 70, a guide wire lumen tube 40 disposed inside the drive shaft 20, and a handle 90.

As illustrated in FIGS. 2 and 3, the outer tubular shaft 50 includes an outer layer 51, an inner layer 52, a shaft distal portion 53, a first covering tube 54 in close contact with the outer peripheral surface of the outer layer 51, and a second covering tube 55 in close contact with the outer peripheral surface of the shaft distal portion 53.

The drive shaft 20 is a multilayer coil in which coils are layered. The drive shaft 20 is flexible and has a property of transmitting a drive torque applied from the proximal side to the distal side. The drive shaft 20 is rotatable inside the outer tubular shaft 50. The drive shaft 20 includes an inner coil 21, an outer coil 22 surrounding the outside of the inner coil 21, and a distal protecting tube 23 that covers the outer peripheral surface of the outer coil 22 at the distal end section. The drive shaft 20 may be a multilayer coil including three or more layers.

The outer coil 22 is wound in a direction in which the helix of a wire material constituting the outer coil 22 is tightened to reduce the diameter when the drive shaft 20 rotates in a predetermined rotation direction (rotation direction of the drive shaft 20 when the medical device 10 is used for cutting and conveyance) in response to a drive torque from the proximal side. That is, the wire material of the outer coil 22 is wound in the direction opposite to the predetermined rotation direction toward the distal side when viewed from the proximal side. The outer coil 22 has characteristics of extending along the axis of the outer coil 22 while decreasing in diameter in response to the drive torque from the proximal side during rotation of the drive shaft 20 in the predetermined rotation direction. The outer coil 22 may be a single-wire coil formed by winding a single wire material or a multi-wire coil formed by winding multiple wire materials.

The inner coil 21 is wound in a direction in which the helix of a wire material constituting the inner coil 21 is loosened to increase the diameter when the drive shaft 20 rotates in the predetermined rotation direction in response to a drive torque from the proximal side. That is, the wire material of the inner coil 21 is wound in the predetermined rotation direction toward the distal side when viewed from the proximal side. The inner coil 21 has characteristics of contracting along the axis of the inner coil 21 while increasing in diameter in response to the drive torque from the proximal side during rotation of the drive shaft 20 in the predetermined rotation direction. The inner coil 21 may be a single-wire coil formed by winding a single wire material or a multi-wire coil formed by winding multiple wire materials.

The outer coil 22 is disposed in close contact with the outer peripheral surface of the inner coil 21. Therefore, when the drive shaft 20 rotates in the predetermined rotation direction, the inner coil 21 is going to contract while increasing in diameter, and the outer coil 22 is going to extend while reducing in diameter, so that the displacement of the outer coil 22 and the inner coil 21 in the radial direction and the axial direction is canceled out. Thus, the multilayer coil including the outer coil 22 and the inner coil 21 can reduce deformation in the radial direction and the axial direction when the drive shaft 20 rotates in the predetermined rotation direction. The action of the outer coil 22 is exerted more strongly than the action of the inner coil 21, because the inner coil 21 has a smaller coil radius than the outer coil 22. Therefore, the drive shaft 20 slightly contracts in the radial direction and slightly extends in the axial direction when the drive shaft 20 rotates in the predetermined rotation direction.

As an example, the outer coil 22 is a multi-wire coil formed by helically and tightly winding sixteen wire materials or metallic materials. The inner coil 21 is a multi-wire coil formed by helically and tightly winding eight wire materials. The number of wire materials of the outer coil 22 and the inner coil 21 is not particularly limited. The cross-sectional shape of the wire material of each of the outer coil 22 and the inner coil 21 is, for example, circular, but is not particularly limited thereto, and may be elliptical, rectangular, square, triangular, or the like.

The drive shaft 20 may be a single-layer coil. In this case, when the wire material of the single-layer coil is wound in the direction opposite to the predetermined rotation direction toward the distal side as viewed from the proximal side, the drive shaft 20 extends along the axis while reducing in diameter in response to the drive torque from the proximal side. When the wire material of the single-layer coil is wound in the predetermined rotation direction toward the distal side as viewed from the proximal side, the drive shaft 20 contracts along the axis while increasing in diameter in response to the drive torque from the proximal side.

The drive shaft 20 has a ring-shaped integrated portion 24 on the proximal side with respect to the shaft distal portion 53. The integrated portion 24 is formed by melting and joining the wire materials arranged in the circumferential direction of the outer coil 22 by welding and integrating the joined wire materials in the circumferential direction. As shown in FIG. 3, adjacent windings of the wire materials are thus integrated to form the ring-shaped or annular integrated portion 24. In the integrated portion 24, not only the outer coil 22 but also at least an outer part of the inner coil 21 is preferably integrated, but the configuration is not limited to this construction. The integrated portion 24 reduces the flexibility of the drive shaft 20, and thus, is preferably not too long in the axial direction.

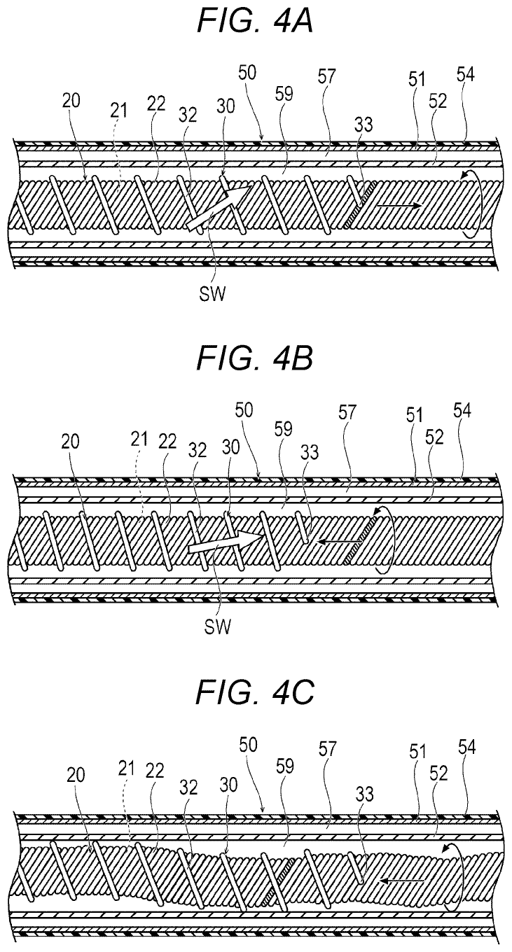

As illustrated in FIGS. 3 and 4A-4C, the conveyance coil 30 is disposed so as to wrap around (helically wrap around) the outer periphery of the drive shaft 20. The conveyance coil 30 is partially in contact with the outer peripheral surface of the drive shaft 20, but may be placed with a clearance. The conveyance coil 30 is formed by loosely winding the wire material constituting the conveyance coil 30 at intervals along the axial direction of the conveyance coil 30. That is, as shown in FIG. 3, the conveyance coil 30 is loosely wound in a helical manner with adjacent helical windings axially spaced apart from one another. The conveyance coil 30 includes a fixed portion 31 which is positioned at a distal end of the conveyance coil 30 and is fixed to the drive shaft 20, and a non-fixed portion 32 which is positioned on the proximal side with respect to the fixed portion 31 and is not fixed to the conveyance coil 30. A coil proximal end 33 located at the proximal end of the conveyance coil 30 is formed in a hemispherical shape. That is, the coil proximal end 33 is formed to have a curved surface. The fixed portion 31 is fixed to the integrated portion 24 of the drive shaft 20 by welding or the like. In a case where the section of the drive shaft 20 to which the fixed portion 31 is fixed is not the integrated portion 24 but any of the wire materials, the wire materials may be excessively melted and broken by welding or the like, so that the drive shaft 20 may be damaged. In addition, in a case where a torque load is applied and stress concentrates on the drive shaft 20 formed of a plurality of thin strands, problems such as breakage and deformation of the strands may occur. On the other hand, when the fixed portion 31 of the conveyance coil 30 is fixed to the integrated portion 24 of the drive shaft 20, which is formed by integrating a plurality of wire materials, by welding or the like, the wire materials are not broken, and damage to the drive shaft 20 can be suppressed.

Note that the fixed portion 31 may be located on at least a part of a distal end section (section within a predetermined range on the distal side) of the conveyance coil 30 instead of the distal end of the conveyance coil 30.

The conveyance coil 30 functions as an Archimedean screw (screw pump) when the drive shaft 20 rotates in the predetermined rotation direction, and conveys a liquid or an object in the proximal direction. To this end, the conveyance coil 30 is wound in the predetermined rotation direction toward the distal side when viewed from the proximal side. The distal end of the conveyance coil 30 is located on the proximal side with respect to the shaft distal portion 53 located at the distal end part of the outer tubular shaft 50. Thus, it is possible to prevent the conveyance coil 30 and the shaft distal portion 53 from being damaged by friction due to the rotation of the drive shaft 20. The conveyance coil 30 may be wound in a direction opposite to the predetermined rotation direction toward the distal side when viewed from the proximal side. In that case, the conveyance coil 30 functions as an Archimedean screw (screw pump) when the drive shaft 20 rotates in the predetermined rotation direction, and can convey liquid or an object in the distal direction.

Examples of materials that can be preferably used for the drive shaft 20 and the conveyance coil 30 include stainless steel, nitinol (NiTi), Ta, Ti, Pt, Au, W, polyolefin such as polyethylene and polypropylene, polyamide, polyester such as polyethylene terephthalate, fluorine-based polymer such as ethylene-tetrafluoroethylene copolymer (ETFE), polyether ether ketone (PEEK), and polyimide.

As illustrated in FIGS. 2 and 3, the distal protecting tube 23 is a tubular body that covers the outer peripheral surface of the drive shaft 20 on the distal side with respect to the conveyance coil 30. The distal protecting tube 23 is disposed inside the shaft distal portion 53 provided on the outer tubular shaft 50. The distal protecting tube 23 is formed of, for example, a heat shrinkable tube that is reduced in diameter by heating to be in close contact with the drive shaft 20. The distal protecting tube 23 prevents the drive shaft 20 and the shaft distal portion 53 from coming into contact with each other and being damaged by the rotation of the drive shaft 20.

The material of the heat-shrinkable tube is not particularly limited, and is, for example, polyolefin, polyamide, Pebax, polyurethane, or polyethylene terephthalate.

The guide wire lumen tube 40 is a tubular body disposed inside the drive shaft 20 as illustrated in FIG. 2. The guide wire lumen tube 40 is formed with a guide wire lumen 41 through which a guide wire passes. The guide wire lumen tube 40 prevents the guide wire passing through the guide wire lumen 41 from rubbing against the drive shaft 20. The distal end part of the guide wire lumen tube 40 protrudes further to the distal side with respect to the drive shaft 20 and is disposed inside the rotation shaft 70 and the cutter 80. The proximal end part of the guide wire lumen tube 40 is connected to the handle 90.

As illustrated in FIGS. 1 to 4C, the outer tubular shaft 50 is a long (elongated) tubular body that accommodates the drive shaft 20. The outer tubular shaft 50 can transmit a torque exerted by an operator on a rotary operation portion 91 fixed to the proximal end part of the outer tubular shaft 50. A first lumen 57 for delivering a liquid such as physiological saline to the distal side is formed between the outer layer 51 and the inner layer 52. At least one side hole 58 penetrating from the inner peripheral surface to the outer peripheral surface is formed at the distal end part of the outer layer 51. The cutter 80 can be oriented to the lesion by rotating the outer tubular shaft 50. The outer tubular shaft 50 has a proximal end opening 120 that opens inside the handle 90 at the proximal end.

It is preferable that the outer layer 51 has flexibility so as to bend in the body lumen and has high torque transmission. Examples of materials that can be used for the outer layer 51 include a material obtained by forming a helical slit or groove by laser processing in a circular tube made of a metal material or a resin material having a certain degree of strength. The material of the outer layer 51 is not particularly limited, and examples thereof that can be preferably used include a metal material such as stainless steel, nitinol (NiTi), Ta, Ti, Pt, Au, and W, and engineering plastics such as ABS resin, polycarbonate (PC), polymethyl methacrylate (PMMA), polyacetal (POM), polyphenyl sulfone (PPSU), polyethylene (PE), carbon fiber, or polyether ether ketone (PEEK).

The first covering tube 54 is a tubular body that is in close contact with the outer peripheral surface of the outer layer 51. The first covering tube 54 suppresses leakage of the liquid in the first lumen 57 from a gap of the helical slit formed in the outer layer 51. The first covering tube 54 is formed of, for example, a heat shrinkable tube that is reduced in diameter by heating to be in close contact with the outer layer 51.

The inner layer 52 is disposed on the inner side of the outer layer 51 with a clearance therebetween. The clearance between the inner layer 52 and the outer layer 51 is the first lumen 57. A second lumen 59 for discharging an object such as a cut thrombus in the proximal direction is formed inside the inner layer 52. The distal end part of the inner layer 52 is fixed to an inner peripheral surface of the shaft distal portion 53 with an adhesive. The proximal end part of the inner layer 52 is located inside the handle 90.

The resin material forming the inner layer 52 desirably has a certain degree of flexibility and low friction, and examples of the material that can be preferably used include polyether ether ketone (PEEK), fluorine-based polymer such as PTFE or ETFE, polymethyl methacrylate (PMMA), polyethylene (PE), polyether block acid copolymer (PEBAX), polyamide, polyimide, and a combination thereof. The inner layer 52 may have a braided reinforcement line. The inner surface of the inner layer 52 is preferably formed of a resin material. This configuration can reduce friction between the inner layer 52 and the conveyance coil 30. As the resin material forming the inner surface of the inner layer 52, polyimide, PTFE, polyamide, or the like can be used. This makes it possible to improve the lubricity while improving the wear resistance of the inner layer 52 and the conveyance coil 30.

As illustrated in FIGS. 2 and 3, the shaft distal portion 53 is located at the distal end part of the outer tubular shaft 50. The shaft distal portion 53 is bent at two bending portions 56 such that the axis of the shaft distal portion 53 at the proximal end part and the axis at the distal end part are shifted from each other. The number of the bending portions 56 may be one or three or more. By rotating the outer tubular shaft 50, the shaft distal portion 53 can orient the cutter 80 toward the lesion and further strongly press the cutter 80 against the lesion. As the material of the shaft distal portion 53, a material like those examples discussed above for the outer layer 51 can be applied, for example.

The second covering tube 55 is a tubular body that is in close contact with the outer peripheral surface of the shaft distal portion 53. The second covering tube 55 is formed of, for example, a heat shrinkable tube that is reduced in diameter by heating to be in close contact with the shaft distal portion 53.

As illustrated in FIG. 2, the distal bearing 60 is fixed to the distal end part of the shaft distal portion 53 and protrudes to the distal side beyond the shaft distal portion 53. The distal bearing 60 supports the rotation shaft 70 fixed to the distal end part of the outer tubular shaft 50 in a rotatable manner. The distal bearing 60 is fixed to the distal end part of the shaft distal portion 53. The distal bearing 60 has, at the distal end, a distal end opening 61 through which an object such as a cut thrombus, blood, and liquid discharged through the side hole 58 is conveyed and delivered into the second lumen 59. The distal end of the distal bearing 60 is positioned proximal to the cutter 80.

The rotation shaft 70 is rotatably supported by the distal bearing 60 fixed to the distal end part of the outer tubular shaft 50. The proximal end part of the rotation shaft 70 is fixed to the distal end part of the drive shaft 20, and the distal end part of the rotation shaft 70 is fixed to the cutter 80. The rotation shaft 70 is formed with at least one groove-shaped passage 71 extending along the axis (extending in the axial direction). The passage 71 allows an object cut by the cutter 80 to pass in the proximal direction through the inside of the distal bearing 60.

The cutter 80 is a member for cutting and reducing the size of an object such as a thrombus, plaque, and a calcified lesion. Therefore, the term “cut” represents the action of applying a force to an object in contact with the cutter 80 and reducing the size of the object. A method for applying a force during cutting and the shape or type of the cut object are not limited. The cutter 80 is strong enough to cut the above-mentioned object. The cutter 80 is fixed to the outer peripheral surface of the rotation shaft 70 at the distal end part. The cutter 80 has a large number of fine abrasive grains on the surface. Alternatively, the cutter 80 may have a sharp blade.

It is preferable that the material of the cutter 80 is strong enough to cut thrombus, and examples of the material that can be preferably used include stainless steel, Ta, Ti, Pt, Au, W, a shape memory alloy, a supersteel alloy, and a ceramic material. The materials of the distal bearing 60 and the rotation shaft 70 preferably have a certain degree of strength, and the above-mentioned materials applicable to the cutter 80 can be used.

The handle 90 includes a rotary operation portion 91, a casing 92, a drive unit 93, an intake port 95, and a liquid delivery port 96 as illustrated in FIG. 1.

The rotary operation portion 91 is connected to the distal end part of the casing 92 in a rotatable manner. In the casing 92, an intake space 121 communicating with the intake port 95 and a liquid delivery space 122 communicating with the liquid delivery port 96 are formed. The proximal end opening 120 of the outer tubular shaft 50 is located in the intake space 121 in a rotatable manner.

The rotary operation portion 91 is a section operated by the operator with his/her finger to apply a torque to the outer tubular shaft 50. The rotary operation portion 91 is rotatably connected to the distal end part of the casing 92. The rotary operation portion 91 is fixed to the outer peripheral surface of the outer tubular shaft 50 at the proximal end part.

The drive unit 93 is, for example, a hollow motor. The drive unit 93 is rotated by a battery (not illustrated) or power supplied from the outside. The drive shaft 20 is fixed to a hollow drive rotor of the hollow motor. The drive unit 93 is not particularly limited in rotation speed and may rotate at, for example, 10,000 to 150,000 rpm, preferably 20,000 to 120,000 rpm. The configuration of the drive unit 93 is not particularly limited. Since the drive unit 93 is a hollow motor, the guide wire lumen tube 40 and the guide wire can pass through the drive unit 93.

The intake port 95 conveys an object, liquid, or the like inside the intake space 121 to the outside. An intake drive source such as a pump or a syringe can also be connected to the intake port 95. From the liquid delivery port 96, a fluid can be delivered to the first lumen 57 of the outer tubular shaft 50 via the liquid delivery space 122.

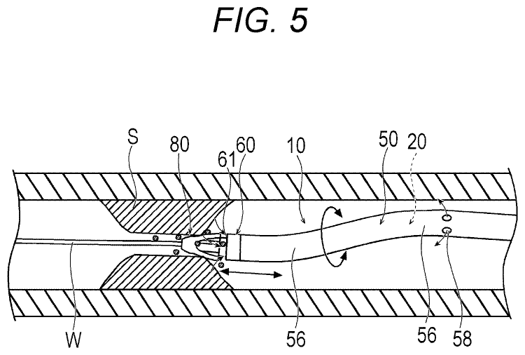

Next, a method of using the medical device 10 according to the embodiment will be described. Here, a case where a calcified lesion S in the blood vessel is broken up and conveyed as illustrated in FIG. 5 will be described as an example.

First, the operator inserts the guide wire W into the blood vessel and brings (moves) the guide wire W to the vicinity of the lesion S. Next, the operator inserts the proximal end of the guide wire W into the guide wire lumen 41 of the medical device 10. Thereafter, the operator moves the cutter 80 of the medical device 10 to the vicinity of the lesion S using the guide wire W as a guide.

Next, the operator operates the handle 90 to start the operation of the drive unit 93. Thus, the drive shaft 20 connected to the drive unit 93, and the rotation shaft 70 and the cutter 80 fixed to the drive shaft 20 rotate. Accordingly, the operator can cut the lesion S by the cutter 80. When the drive shaft 20 rotates, the conveyance coil 30 fixed to the drive shaft 20 generates a force for conveying the liquid or the object in the second lumen 59 to the proximal side as illustrated in FIGS. 3 and 4A. Thus, the cut fragment(s) enters through the distal end opening 61 of the second lumen 59 of the outer tubular shaft 50 and is discharged as illustrated in FIG. 2.

The operator can change the position of the cutter 80 in the circumferential direction by operating the rotary operation portion 91 illustrated in FIG. 1. When the operator turns the rotary operation portion 91, the outer tubular shaft 50 fixed to the rotary operation portion 91 rotates. When the outer tubular shaft 50 rotates, the position and direction of the section of the outer tubular shaft 50 distal to the bending portion 56 change, so that the position and direction of the cutter 80 can be changed, as illustrated in FIG. 5. Therefore, the operator can perform the cutting operation while changing the position and direction of the cutter 80 only by operating the rotary operation portion 91 without entirely rotating the handle 90 that is difficult to rotate greatly. Further, the operator moves the outer tubular shaft 50 back and forth along the longitudinal direction of the blood vessel by moving the entire handle 90 or the outer tubular shaft 50 exposed to the outside of the body. As a result, the lesion S can be cut along the longitudinal direction of the blood vessel by the cutter 80.

When a fluid is fed from the liquid delivery port 96 to the first lumen 57 of the outer tubular shaft 50 via the liquid delivery space 122, the fluid is discharged to the first lumen 57 as illustrated in FIG. 3. The physiological saline delivered to the first lumen 57 moves in the distal direction and is released into the blood vessel through the side hole 58 formed at the distal end part of the outer layer 51.

A portion of the physiological saline released into the blood vessel enters the second lumen 59 together with blood and the cut object(s) through the distal end opening 61 of the outer tubular shaft 50 as illustrated in FIGS. 2 and 5. The object(s) and the liquid that have entered the second lumen 59 move in the second lumen 59 in the proximal direction. The object(s) and the liquid that have entered the second lumen 59 are conveyed in the proximal direction by the conveyance coil 30 and discharged to the outside through the intake port 95 of the handle 90 as illustrated in FIG. 1.

The rotating drive shaft 20 and the conveyance coil 30 receive various loads. The drive shaft 20 in the present embodiment contracts in the radial direction and extends in the axial direction when rotating in the predetermined rotation direction as illustrated in FIG. 4A. On the other hand, the conveyance coil 30 is fixed to the drive shaft 20 only at the fixed portion 31 on the distal side, and has the non-fixed portion 32 not fixed to the drive shaft 20 at the proximal end part. As shown in FIG. 3, the fixed portion 31 of the conveyance coil 30, which is fixed to the drive shaft 20 and which is the only part of the conveyance coil 30 fixed to the drive shaft 20, is the distal-most end portion or section of the conveyance coil 30 in the distal half of the conveyance coil 30. Therefore, the conveyance coil 30 is less likely to receive a force to extend in the axial direction due to contraction of the drive shaft 20 in the radial direction. On the other hand, the conveyance coil 30 receives a drive torque at the fixed portion 31 on the distal side from the drive shaft 20 that rotates in a predetermined rotation direction. As a result, independently of the extension of the drive shaft 20, the conveyance coil 30 is going to extend in the axial direction while reducing in diameter due to the helix of the wire materials constituting the conveyance coil 30 being tightened. Further, the conveyance coil 30 applies a conveying force in the proximal direction to the liquid or the object in the second lumen 59 to receive a reaction force in the distal direction from the liquid or the object, so that the helical pitch width of the conveyance coil 30 is further reduced as illustrated in FIG. 4B, for example. When the helical pitch width is reduced, the generation of a swirl flow SW (swirl) of the fluid that receives a force from the rotating conveyance coil 30 is reduced, whereby the conveyance efficiency is improved. As described above, the conveyance coil 30 receives various forces depending on the situation, and extends or contracts along the axis. Moreover, since the conveyance coil 30 is not a tightly wound coil but is a loosely wound coil, the conveyance coil 30 is easy to extend and contract along the axis (axial direction). In this manner, the conveyance coil 30 extends or contracts in the axial direction independently of the drive shaft 20 on the proximal side with respect to the fixed portion 31, and thus, moves relative to the drive shaft 20 surrounded by the non-fixed portion 32 of the conveyance coil 30.

In response to the drive torque from the proximal side, the drive shaft 20 contracts in the radial direction and extends in the axial direction. At this time, the drive shaft 20 is twisted by receiving a torque load from, for example, the cutter 80 on the distal side with respect to the drive shaft 20, and is curved so as to meander in the outer tubular shaft 50 as illustrated in FIG. 4C, so that the drive shaft 20 is likely to be shorter than the outer tubular shaft 50 in the axial direction. On the other hand, the conveyance coil 30 comes into contact with the inner peripheral surface of the outer tubular shaft 50, and thus is going to stay at the current position due to friction with the outer tubular shaft 50. Therefore, on the proximal side with respect to the fixed portion 31, the drive shaft 20 surrounded by the non-fixed portion 32 of the conveyance coil 30 relatively moves to the distal side with respect to the non-fixed portion 32.

As described above, even when the drive shaft 20 and the conveyance coil 30 are deformed under various loads, the non-fixed portion 32 of the conveyance coil 30 can change the relative position in the axial direction with respect to the drive shaft 20. Therefore, the load on the fixed portion 31 of the conveyance coil 30 with respect to the drive shaft 20 is reduced, and detachment of the conveyance coil 30 from the drive shaft 20 is suppressed. In addition, it is possible to suppress, for example, a phenomenon in which the wire materials of the conveyance coil 30 overlay each other and a situation in which the winding direction of the conveyance coil 30 is reversed.

After the cutting and conveyance of the lesion S are completed, the operator operates the handle 90. Thus, the rotation of the drive shaft 20 is stopped, and the liquid delivery is stopped. Thereafter, the operator removes the medical device 10 from the blood vessel, and the treatment is completed.

As described above, the medical device 10 according to the present embodiment is the medical device 10 that removes an object in a body lumen, and includes a drive shaft 20 that is rotatable, a cutter 80 that is rotationally driven by the drive shaft 20 and is able to cut the object, an outer tubular shaft 50 that rotatably accommodates the drive shaft 20, and a conveyance coil 30 that is loosely wound and is disposed to wrap around the outer periphery of the drive shaft 20, and the conveyance coil 30 is fixed to the drive shaft 20 only at a distal end section of the conveyance coil 30. With this configuration, in the medical device 10, when the drive shaft 20 extends and contracts in the axial direction, a section of the conveyance coil 30 proximal to the distal end section fixed to the drive shaft 20 can move in the axial direction relative to the drive shaft 20, so that a load on the fixed portion 31 of the conveyance coil 30 with respect to the drive shaft 20 can be reduced. Therefore, the medical device 10 can prevent detachment of the conveyance coil 30 fixed to the drive shaft 20 from the drive shaft 20.

During rotation, the conveyance coil 30 contracts, and the drive shaft 20 extends. Due to the contraction of the conveyance coil 30, it is possible to prevent the fluid that receives a force from the conveyance coil 30 from generating the swirl flow SW, whereby the conveyance efficiency can be improved. Due to the extension of the drive shaft 20, it is possible to suppress occurrence of a defect (for example, a defect of the drive shaft 20 being stuck in the outer tube shaft 50) due to a decrease in length of the drive shaft 20.

The conveyance coil 30 is fixed to the drive shaft 20 only at the distal end of the conveyance coil 30. With this configuration, the load on the fixed portion 31 of the conveyance coil 30 with respect to the drive shaft 20 can be further reduced, whereby the detachment of the conveyance coil 30 from the drive shaft 20 can be effectively suppressed. In addition, the flexibility of the drive shaft 20 and the conveyance coil 30 is improved, and the operability of the medical device 10 in the curved body is improved.

The conveyance coil 30 includes a multi-wire coil formed by winding a plurality of wire materials, includes an integrated portion 24 formed by melting and integrating a plurality of wire materials arranged in a circumferential direction over the entire circumference, and is fixed to the integrated portion 24. When the section of the drive shaft 20 to which the conveyance coil 30 is fixed is not the integrated portion 24 but any of the wire materials, the wire materials may be broken. On the other hand, when the conveyance coil 30 is fixed to the integrated portion 24 of the drive shaft 20, damage to the drive shaft 20 can be suppressed.

The coil proximal end 33 located at the proximal end of the conveyance coil 30 is formed to have a curved surface. This configuration can prevent damage to the drive shaft 20 and the outer tubular shaft 50 with which the coil proximal end 33 of the conveyance coil 30 is in contact.

The medical device is not limited to the above embodiment, and various modifications may be made by those skilled in the art within the technical idea of the present invention. For example, the body lumen into which the medical device 10 is inserted is not limited to a blood vessel, and may be, for example, a vascular channel, a ureter, a bile duct, a fallopian tube, a hepatic duct, or the like. In addition, the medical device can also be used for a surgical instrument such as a morcellator.

The detailed description above describes embodiments of a medical device and operational method representing examples of the new medical device and method disclosed here. The invention is not limited, however, to the precise embodiments and variations described. Various changes, modifications and equivalents can be effected by one skilled in the art without departing from the spirit and scope of the invention as defined in the accompanying claims. It is expressly intended that all such changes, modifications and equivalents that fall within the scope of the claims are embraced by the claims.

What is claimed is:

1. A medical device that removes an object in a body lumen, the medical device comprising:
a drive shaft that is rotatable;
a cutter that is rotationally driven by the drive shaft and is able to cut the object;
an outer tubular shaft that rotatably accommodates the drive shaft;
a conveyance coil that is loosely wound and is disposed to wrap around an outer periphery of the drive shaft; and
the conveyance coil being fixed to the drive shaft only at a distal end section of the conveyance coil.

2. The medical device according to claim 1, wherein, during rotation, the conveyance coil extends along an axial direction, and the drive shaft extends along the axial direction.

3. The medical device according to claim 1, wherein the conveyance coil is fixed to the drive shaft only at a distal end of the conveyance coil.

4. The medical device according to claim 1, wherein
the drive shaft includes a multi-wire coil comprised of plural wound wire materials, the drive shaft including an integrated portion in which a plurality of the wire materials arranged in a circumferential direction over an entire circumference are melted and integrated with one another, and
the distal end section of the conveyance coil being fixed to the integrated portion.

5. The medical device according to claim 1, wherein a coil proximal end located at a proximal end of the conveyance coil has a curved surface.

6. A medical device operable to remove an object in a body lumen, the medical device comprising:
a rotatable drive shaft that extends in an axial direction and that has a distal end portion, the rotatable drive shaft having an outer periphery;
a cutter connected to the drive shaft so that rotation of the drive shaft results in rotation of the cutter, the cutter being located at the distal end portion of the drive shaft and being configured to cut the object when the cutter is brought into contact with the object while rotating;
an outer tubular shaft having an interior in which is located the drive shaft, the outer tubular shaft having an inner periphery facing the outer periphery of the drive shaft so that a space exists between the inner periphery of the outer tubular shaft and the outer periphery of the drive shaft;
an opening through which is conveyable a part of the object cut by the cutter and liquid in the body lumen, the opening being in communication with the space between the inner periphery of the outer tubular shaft and the outer periphery of the drive shaft so that the part of the object cut by the cutter and the liquid can enter the space by way of the opening;
a conveyance coil helically wound around the outer periphery of the drive shaft so that axially adjacent windings of the conveyance coil are axially spaced apart from one another, the conveyance coil being located in the space, the conveyance coil having a distal end portion terminating at a distal-most end of the conveyance coil and a proximal end portion terminating at a proximal-most end of the conveyance coil; and
at least a part of the distal end portion of the conveyance coil being fixed to the drive shaft so that rotation of the drive shaft results in rotation of the conveyance coil so that the conveyance coil conveys the part of the object cut by the cutter and the liquid in the axial direction, an entirety of the proximal end portion of the conveyance coil being a non-fixed portion of the conveyance coil that is not fixed to the drive shaft so that the drive shaft is movable relative to the non-fixed portion of the conveyance coil.

7. The medical device according to claim 6, wherein the opening is a distal end opening located at a distal end of the outer tubular shaft and proximal of a proximal end of the cutter.

8. The medical device according to claim 6, wherein the conveyance coil is helically wound around an intermediate portion of the drive shaft so that one portion of the drive shaft extends distally beyond the distal-most end of the conveyance coil and an other portion of the drive shaft extends proximally beyond the proximal-most end of the conveyance coil.

9. The medical device according to claim 6, wherein the drive shaft is comprised of at least one wire that is helically wound in a coil so that adjacent helical windings of the at least one wire are in contact with one another.

10. The medical device according to claim 6, wherein the drive shaft includes an annular integrated portion in which adjacent windings of the at least one wire are joined together by melting, the at least a part of the distal end portion of the conveyance coil being fixed to the annular integrated portion of the drive shaft.

11. The medical device according to claim 6, wherein the drive shaft is comprised of: i) at least a first wire that is helically wound to form an outer coil so that adjacent helical windings of the at least first wire are in contact with one another; and ii) at least a second wire that is helically wound to form an inner coil so that adjacent helical windings of the at least second wire are in contact with one another; iii) the outer col surrounding the inner coil.

12. The medical device according to claim 6, wherein the outer tubular shaft is comprised of an inner layer and an outer layer, the inner layer being radially inwardly of the outer layer, the inner layer having an outer surface and the outer layer having an inner surface, the outer surface of the inner layer and the inner surface of the outer layer facing one another and being spaced apart from one another so that a lumen exist between the outer surface of the inner layer and the inner surface of the outer layer.

13. The medical device according to claim 12, wherein the outer layer includes at least one through hole that opens into the lumen between the outer surface of the inner layer and the inner surface of the outer layer.

14. The medical device according to claim 12, wherein the inner layer extends distally beyond the distal-most end of the conveyance coil and extends proximally beyond a proximal-most end of the conveyance coil.

15. The medical device according to claim 6, wherein the drive shaft is comprised of at least one wire that is helically wound as a drive shaft coil so that adjacent helical windings of the at least one wire are in contact with one another, the drive shaft also including a distal protecting tubular body covering an outer peripheral surface of a distal portion of the drive shaft coil, the distal protecting tubular body being spaced distally from the distal-most end of the conveyance coil.

16. The medical device according to claim 15, wherein the outer tubular shaft is comprised of an inner layer and an outer layer, the inner layer having an outer surface and the outer layer having an inner surface, the outer surface of the inner layer and the inner surface of the outer layer facing one another, the outer tubular shaft also comprising a tubular shaft distal portion having a proximal-most end portion that axially overlaps with and is fixed to a distal-most end portion of the inner layer, the distal protecting tubular body having an outer surface that faces an inner surface of the tubular shaft distal portion.

17. The medical device according to claim 16, wherein the proximal-most end portion tubular shaft distal portion extends proximally beyond a proximal-most end of the distal protecting tubular body.

18. The medical device according to claim 6, further comprising a handle, the drive shaft including a proximal portion operatively connected to a drive unit that, when operated, rotates the drive shaft, the drive unit being located in the handle.

19. A method comprising:
positioning a cutter adjacent an object in a lumen in a living body, the cutter being connected to a drive shaft, the drive shaft being positioned in an outer tubular shaft, a conveyance coil wound around an outer periphery of the drive shaft so that adjacent windings of the conveyance coil are spaced apart from one another, the conveyance coil having a distal end section and a proximal end section, the conveyance coil being fixed to the drive shaft only at the distal end section of the conveyance coil so that an entirety of the proximal end section of the conveyance coil is a non-fixed portion of the conveyance coil that is not fixed to the drive shaft;
rotating the cutter through rotation of the drive shaft while the cutter is in contact with the object to cut the object, the rotation of the drive shaft resulting in rotation of the conveyance coil; and
the non-fixed portion of the conveyance coil that is proximal to the distal end section being movable in an axial direction relative to the drive shaft during the rotating of the cutter through the rotation of the drive shaft.

20. The method according to claim 19, wherein the cutting of the object produces cut parts of the object, and further comprising conveying liquid and the cut parts of the object by the conveyance coil during the rotation of the conveyance coil that operates as an Archimedean screw.
